(19) United States
(12) Patent Application Publication
(10) Pub. No.: US 2023/0386504 A1
(43) Pub. Date: Nov. 30, 2023

(54) SYSTEM AND METHOD FOR PATHOLOGICAL VOICE RECOGNITION AND COMPUTER-READABLE STORAGE MEDIUM

(71) Applicant: National Yang Ming Chiao Tung University, Taipei (TW)

(72) Inventors: Oscar Kuang-Sheng Lee, Taipei (TW); Hao-Chun Hu, Taipei (TW); Si-Han Wang, Taipei (TW)

(21) Appl. No.: 17/826,215

(22) Filed: May 27, 2022

(51) Int. Cl. G10L 25/66; G10L 15/06; G10L 15/22; G10L 25/24; G10L 25/18; A61B 5/00

(52) U.S. Cl. CPC G10L 25/66; G10L 15/063; G10L 15/22; G10L 25/24; G10L 25/18; A61B 5/4803; A61B 5/7267; A61B 5/7275

(57) ABSTRACT

A system and a method for pathological voice recognition and a computer-readable storage medium are provided. The method for pathological voice recognition comprises: capturing a voice signal; processing the voice signal using Mel Frequency Cepstral Coefficients (MFCC) algorithm to obtain an MFCC spectrogram; extracting features from the MFCC spectrogram; and predicting a pathological condition of the voice signal based on the features of the MFCC spectrogram of the voice signal by a deep learning model, the pathological condition of the voice signal including normal, unilateral vocal paralysis, adductor spasmodic dysphonia, vocal atrophy, and organic vocal fold lesions.

### TECHNICAL FIELD

The present disclosure relates to a vocal fold condition prediction through voice recognition, and more particularly, to a pathological condition of the vocal fold using artificial intelligence.

### DESCRIPTION OF RELATED ART

The impact of a voice disorder has been increasingly recognized as a public health concern. Dysphonia influences the quality of physical, social, and occupational aspects of life by interfering with communication. A nationwide insurance claims data analysis of treatment-seeking for dysphonia showed a prevalence rate of 0.98% among 55 million individuals, and this rate reached 2.5% among those older than 70 years. However, the overall dysphonia incidence for the aging population is estimated to be much higher (12%-35%), which may imply that dysphonia is commonly overlooked by patients, resulting in underdiagnosis.

According to the state-of-the-art clinical practice guidelines for dysphonia of the American Academy of Otolaryngology-Head and Neck Surgery Foundation, a laryngoscopic examination is recommended if dysphonia fails to resolve or improve within 4 weeks. A comparison of diagnoses made by primary care physicians and those made by laryngologists and speech-language pathologists with experience in interpreting stroboscopy at multidisciplinary voice clinics indicated that the primary care physicians' diagnoses of dysphonia were different in 45%-70% of cases. However, the laryngoscopic examination is an invasive procedure. To achieve an accurate diagnosis, it must be performed by an experienced laryngologist. The examination equipment is expensive and not generally available in primary care units. In places without sufficient medical resources, delayed diagnoses and treatments are common.

Therefore, a noninvasive diagnostic tool is needed to efficiently screen significant clinical conditions for further evaluation.

### SUMMARY

The present disclosure provides a method for pathological voice recognition, the method comprising: capturing a voice signal; processing the voice signal using Mel Frequency Cepstral Coefficients (MFCC) algorithm to obtain an MFCC spectrogram; extracting features from the MFCC spectrogram; and predicting a pathological condition of the voice signal based on the features of the MFCC spectrogram of the voice signal by a deep learning model.

In an embodiment, the method according to the present disclosure further comprising: capturing a plurality of voice samples into a database; dividing the plurality of voice samples into a training set and a testing set; processing the training set of the plurality of voice samples using Mel Frequency Cepstral Coefficients (MFCC) algorithm to obtain a plurality of MFCC spectrograms; extracting a plurality of features from the plurality of MFCC spectrograms of the training set of the voice samples; and inputting the plurality of features into the deep learning model to train the deep learning model, wherein the plurality of features comprises MFCC spectrogram, delta MFCC spectrogram, and/or second-order delta MFCC spectrogram, wherein each of the plurality of voice samples includes a sustained vowel sound followed by a continuous speech.

In an embodiment, the method according to the present disclosure further comprising: training the deep learning model by classifying the training set of the voice samples into two classifications, three classifications, four classifications, or five classifications.

In one embodiment, the two classifications include normal voices and a group of adductor spasmodic dysphonia, organic vocal fold lesions, unilateral vocal paralysis and vocal atrophy. In another embodiment, the three classifications include normal voices, adductor spasmodic dysphonia, and a group consist of organic vocal fold lesions, unilateral vocal paralysis and vocal atrophy. In yet another embodiment, the four classifications include normal voices, adductor spasmodic dysphonia, organic vocal fold lesions and a group consist of unilateral vocal paralysis and vocal atrophy. In the other embodiment, the five classifications include normal voices, adductor spasmodic dysphonia, organic vocal fold lesions, unilateral vocal paralysis, and vocal atrophy.

In an embodiment, the method according to the present disclosure further comprising: training the deep learning model by adding a dropout function, using minibatches, tuning a learning rate based on cosine annealing and a 1-cycle policy strategy and applying a Softmax layer as an output layer, and assembling the trained deep learning model by average output probability.

In an embodiment, extracting a plurality of features from the plurality of MFCC spectrograms of the training set of the voice samples further comprises: using pre-emphasis, windowing, fast Fourier transform, Mel filtering, nonlinear transformation, and/or discrete cosine transform to extract the plurality of features therefrom, wherein the plurality of features comprises MFCC, delta MFCC, and/or second-order delta MFCC.

The present disclosure further provides a non-transitory computer-readable storage medium storing computer-readable instructions that, when executed, cause a system to perform the above method according to the present disclosure.

The present disclosure provides a system for pathological voice recognition, the system comprising: a transducer configured to capture a voice signal; a processor including a deep learning model, configured to: processing the voice signal using Mel Frequency Cepstral Coefficients (MFCC) algorithm to obtain an MFCC spectrogram; extract features from the MFCC spectrogram; and predict a pathological condition of the voice signal based on the features of the MFCC spectrogram of the voice signal by the deep learning model.

In an embodiment, the system according to the present disclosure further comprising: a database configured to receive a plurality of voice samples captured by the transducer; wherein the processor is configured to: divide the plurality of voice samples into a training set and a testing set; process the training set of the voice samples using Mel Frequency Cepstral Coefficients (MFCC) algorithm to obtain a plurality of MFCC spectrograms; extract a plurality of features from the plurality of MFCC spectrograms of the training set of the voice samples; input the plurality of features into the deep learning model to train the deep learning model, wherein the plurality of features comprises MFCC spectrogram, delta MFCC spectrogram, and/or second-order delta MFCC spectrogram, wherein each of the plurality of voice samples includes a sustained vowel sound followed by a continuous speech.

In an embodiment, the processor of the system is further configured to: train the deep learning model by classifying the training set of the voice samples into two classifications, three classifications, four classifications, or five classifications. In one embodiment, the two classifications include normal voices and a group of adductor spasmodic dysphonia, organic vocal fold lesions, unilateral vocal paralysis, and vocal atrophy. In another embodiment, the three classifications include normal voices, adductor spasmodic dysphonia, and a group consist of organic vocal fold lesions, unilateral vocal paralysis, and vocal atrophy. In yet another embodiment, the four classifications include normal voices, adductor spasmodic dysphonia, organic vocal fold lesions, and a group consist of unilateral vocal paralysis and vocal atrophy. In the other embodiment, the five classifications include normal voices, adductor spasmodic dysphonia, organic vocal fold lesions, unilateral vocal paralysis, and vocal atrophy.

In an embodiment, the processor of the system is further configured to train the deep learning model by adding a dropout function, using minibatches, tuning a learning rate based on cosine annealing and a 1-cycle policy strategy and applying a Softmax layer as an output layer, and assemble the trained deep learning model by average output probability. Further, the processor is further configured to use pre-emphasis, windowing, fast Fourier transform, Mel filtering, nonlinear transformation, and/or discrete cosine transform to extract the plurality of features, wherein the plurality of features comprises MFCC, delta MFCC, and/or second-order delta MFCC.

### BRIEF DESCRIPTION OF THE DRAWINGS

The present disclosure can be more fully understood by reading the following descriptions of the embodiments, with reference made to the accompanying drawings, wherein:

FIG. 1 is a schematic diagram illustrating an exemplifying structure of the system for pathological voice recognition in accordance with embodiments of the present disclosure;

FIG. 2A is a flow chart illustrating exemplifying steps of the method for pathological voice recognition in accordance with embodiments of the present disclosure;

FIG. 2B is a flow chart illustrating exemplifying steps of a training process of a deep learning model for pathological voice recognition in accordance with embodiments of the present disclosure;

FIGS. 3A through 3C are diagram illustrating visual features of normal voice sample after MFCC conversion process;

FIG. 3D is a diagram illustrating the changes of the loss function value over the training and validation sets;

FIGS. 4A through 4D are confusion matrix of 4 conditions of two classifications, three classifications, four classifications, and five classifications, respectively; and

FIGS. 5A through 5D are receiver operating characteristic curves of 4 conditions of two classifications, three classifications, four classifications, and five classifications, respectively.

### DETAILED DESCRIPTION OF THE EMBODIMENTS

The following embodiments are provided to illustrate the present disclosure in detail. A person having ordinary skill in the art can easily understand the advantages and effects of the present disclosure after reading this disclosure, and also can implement or apply in other different embodiments. Therefore, any element or method within the scope of the present disclosure disclosed herein can combine with any other element or method disclosed in any embodiment of the present disclosure.

The proportional relationships, structures, sizes and other features shown in accompanying drawings of this disclosure are only used to illustrate embodiments described herein, such that those with ordinary skill in the art can read and understand the present disclosure therefrom, of which are not intended to limit the scope of this disclosure. Any changes, modifications, or adjustments of said features, without affecting the designed purposes and effects of the present disclosure, should all fall within the scope of technical content of this disclosure.

As used herein, when describing an object “comprises,” “includes” or “has” a limitation, unless otherwise specified, it may additionally encompass other elements, components, structures, regions, parts, devices, systems, steps, connections, etc., and should not exclude others.

As used herein, sequential terms, such as “first,” “second,” etc., are only cited in convenience of describing or distinguishing limitations such as elements, components, structures, regions, parts, devices, systems, etc. from one another, which are not intended to limit the scope of this disclosure, nor to limit spatial sequences between such limitations. Further, unless otherwise specified, wordings in singular forms such as “a,” “an” and “the” also pertain to plural forms, and wordings such as “or” and “and/or” may be used interchangeably.

As used herein, the terms “comprise,” “comprising,” “include,” “including,” “have,” “having,” “contain,” “containing,” or any other variations thereof are intended to cover a non-exclusive inclusion. For example, a composition, mixture, process or method that comprises a list of elements is not necessarily limited to only those elements, but may include other elements not expressly listed, or inherent to such composition, mixture, process, or method.

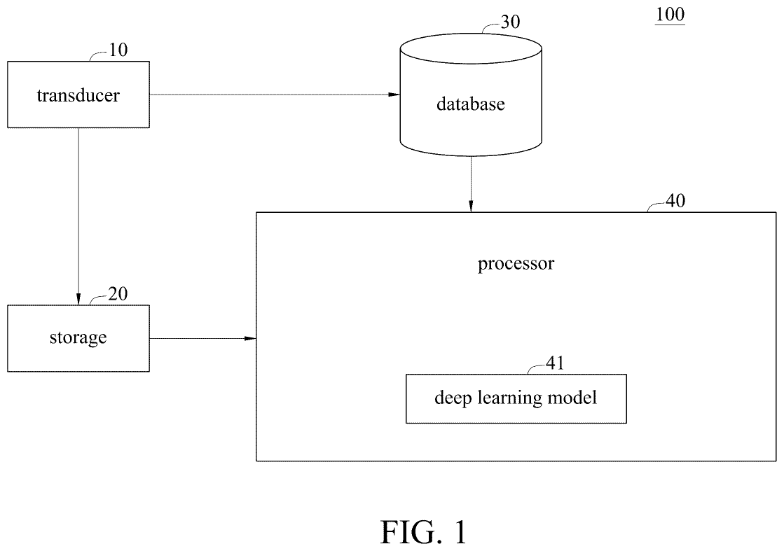

Referring to FIG. 1, there is shown a computer implemented system 100 for pathological voice recognition. The system 100 includes but is not limited to a transducer 10, a storage 20, a database 30, and a processor 40 with a deep learning model 41.

The transducer 10, such as a microphone, is configured to receive or capture voices from people, and converts voice waves into electric current, i.e., voice signal. In an embodiment, the transducer 10 receives voice from a person, then transmits voice signal of the person to the storage 20 for being used to predict a pathological condition of the vocal fold of the person. In another embodiment, the transducer 10 receives voices from a plurality of people, then transmits voice signals into the database 30 for being a plurality of voice samples and being used to train a deep learning model.

In one embodiment, the voice signal or the voice samples, herein, includes a sustained vowel sound followed by a continuous speech.

The processor 40 is configured to analyze and perform Mel Frequency Cepstral Coefficients (MFCC) algorithm and features extraction, and the deep learning model 41 is trained to perform the pathological condition prediction of the voice signal. Specifically, the processor 40 processes the voice signal using MFCC algorithm to obtain an MFCC spectrogram and extracts features from the MFCC spectrogram. The deep learning model 41 predicts predict a pathological condition of the voice signal based on the features of the MFCC spectrogram of the voice signal.

In an embodiment, different CNN architectures, such as EfficientNet-B0 to B6, SENet154, Se_resnext101_32 x4d, and se_resnet152 models, are used.

In one embodiment, these voice samples of the people are divided into a training set and a testing set. Each voice sample in the training set are analyzed and performed MFCC algorithm to obtain an MFCC spectrogram, and then features extraction from the MFCC spectrogram is performed using pre-emphasis, windowing, fast Fourier transform, Mel filtering, nonlinear transformation, and/or discrete cosine transform. And the features are inputted into a first layer of the deep learning model (e.g., CNN model) to train the model, wherein the features comprise MFCC, delta MFCC, and/or second-order delta MFCC. In addition, these voice samples being features extracted are classified into multiple conditions to train the CNN model. In one embodiment, a condition of the two classifications includes normal voices and a group of adductor spasmodic dysphonia, organic vocal fold lesions, unilateral vocal paralysis, and vocal atrophy. In another embodiment, a condition of the three classifications include normal voices, adductor spasmodic dysphonia, and a group consist of organic vocal fold lesions, unilateral vocal paralysis, and vocal atrophy. In yet another embodiment, a condition of the four classification include normal voices, adductor spasmodic dysphonia, organic vocal fold lesions, and a group consist of unilateral vocal paralysis and vocal atrophy. In the other embodiment, a condition of the five classification include normal voices, adductor spasmodic dysphonia, organic vocal fold lesions, unilateral vocal paralysis, and vocal atrophy.

After the deep learning model 41 has been trained, the processer 40 including the deep learning model 41 is configured to perform the prediction for a pathological condition of a vocal fold of a person based on a voice signal from the person, with MFCC and features extractions being performed for the voice signal.

As such, through the voice signal of the person, the pathological condition of the vocal fold of the person is at least one of adductor spasmodic dysphonia, organic vocal fold lesions, unilateral vocal paralysis, and vocal atrophy.

In a case of sample collection and model train, a microphone may be implemented as the transducer 10, and a computer may be implemented as the database 30 and the processor 40. In a case of vocal fold condition prediction, a portable device, such as a smart phone, may be implemented as the transducer 10, and a cloud computing platform may be implemented as the database 30 and the processor 40.

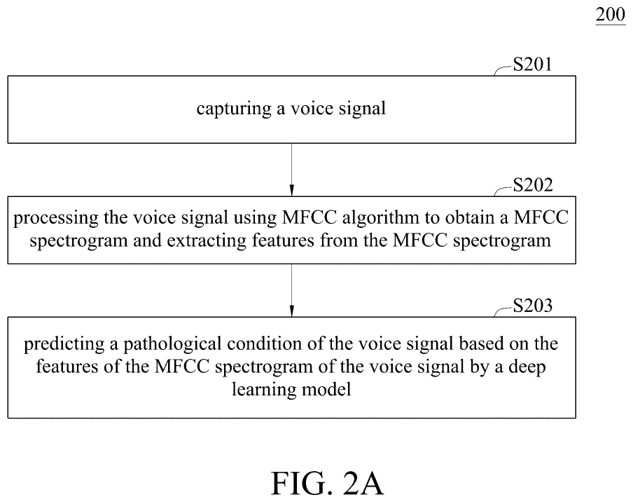

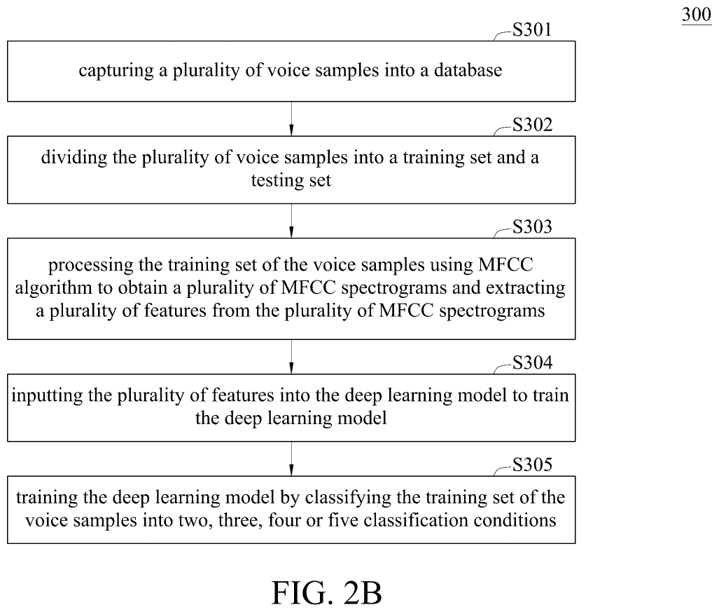

Referring to FIG. 2A, a method 200 of a prediction process of the model of the computer implemented system for pathological voice recognition according to the present disclosure is illustrated.

At step 201, capturing a voice signal.

At step 202, processing the voice signal using MFCC algorithm to obtain a MFCC spectrogram and extracting features from the MFCC spectrogram.

At step 203, predicting a pathological condition of the voice signal based on the features of the MFCC spectrogram of the voice signal by a deep learning model.

Referring to FIG. 2B, a method 300 of a training process of the model of the computer implemented system for pathological voice recognition according to the present disclosure is illustrated.

At step 301, capturing a plurality of voice samples into a database.

At step 302, dividing the plurality of voice samples into a training set and a testing set.

At step 303, processing the training set of the voice samples using MFCC algorithm to obtain a plurality of MFCC spectrograms and extracting a plurality of features from the plurality of MFCC spectrograms of the training set of the voice samples.

At step 304, inputting the plurality of features into a first layer of the deep learning model to train the deep learning model, wherein the plurality of features comprises MFCC, delta MFCC, and/or second-order delta MFCC.

At step 305, training the deep learning model by classifying the training set of the voice samples into two, three, four or five classification conditions.

In some embodiments, a computer readable medium is also present, which stores a computer executable code and/or instructions, and the computer executable code and/or instructions is configured to realize the steps as discussed in this disclosure after being executed.

A detailed description of how working mechanisms of the abovementioned processor is designed will be provided.

Methodology

### Sample Collection

In an embodiment, there are 189 normal voice samples and 552 samples of voice disorders, including vocal atrophy (n=224), unilateral vocal paralysis (n=50), organic vocal fold lesions (n=248), and adductor spasmodic dysphonia (n=30). Voice samples of a sustained vowel sound/a:/ followed by continuous speech of, e.g., a Mandarin passage, are recorded at a comfortable loudness level with a microphone-to-mouth distance of approximately 15-20 cm using a high-quality microphone with a digital amplifier and a 40- to 45-dB background noise level. The sampling rate is 44,100 Hz with 16-bit resolution, and data are saved in an uncompressed .wav format.

### Comparison and Evaluation

In the embodiment, the 741 voice samples are divided into 2 sets: 593 samples for the training set and 148 samples for the testing set. Using, for example, computer-based randomization, 152 of the 189 normal voice samples, 40 of the 50 unilateral vocal paralysis samples, 24 of the 30 adductor spasmodic dysphonia samples, 179 of the 224 vocal atrophy samples, and 198 of the 248 organic vocal fold lesion samples are selected for the training set (see Table 1).

TABLE 1
Details of the voice samples used for experiments (N = 741)

| | | Training set | Test set |
|---|---|---|---|
| | Sample | (n = 593) | (n = 148) |
| Normal (n = 189) | Normal (n = 189) | 152 | 37 |
| Disorders | Unilateral vocal | 40 | 10 |
| (n = 552) | paralysis (n = 50) | | |
| | Adductor spasmodic | 24 | 6 |
| | dysphonia (n = 30) | | |
| | Vocal atrophy (n = 224) | 179 | 45 |
| | Organic vocal fold | 198 | 50 |
| | lesions (n = 248) | | |

To manage the limited size of the training set, a mix-up approach for data augmentation can be used. In an embodiment, the mix-up approach may be performed by utilizing the methodology disclosed in the document “Mixup: beyond empirical risk minimization” by Zhang H, Cisse M, Dauphin Y, Lopez-Paz D. For example, the mix-up approach is applied for audio scene classification using convoluted neural networks (CNNs) to reduce overfitting and obtain higher prediction accuracy. In one embodiment, 2 voice files are randomly selected and then mixed into 1 voice file with randomly selected weights to construct the virtual training examples. Next, each of these voice files is randomly cropped to achieve 10 voice files with a length of 11.88 seconds (plateau point of the training length within the graphics processing unit memory limitations of our hardware, according to our preliminary tests). Additionally, oversampling may be used to adjust the class distribution of the data. In an embodiment, the oversampling may be performed by utilizing the methodology disclosed in the document “A survey of predictive modelling under imbalanced distributions” by Branco P, Torgo L, Ribeiro R.

Next, Mel Frequency Cepstral Coefficients (MFCC) conversion process is performed and analyzed for the above processed voice file to obtain a spectrogram. And feature extraction from MFCC is performed using pre-emphasis, windowing, fast Fourier transform, Mel filtering, nonlinear transformation, and/or discrete cosine transform. In an embodiment, a process to create MFCC features may be performed by utilizing the methodology disclosed in the document “Mel Frequency cepstral coefficients for music modeling” by Logan B. As a result, the first feature consists of 40-dimension MFCCs. In an embodiment, the MFCC features of multidimensional MFCC may be performed by utilizing the methodology disclosed in the documents “Comparison of multidimensional MFCC feature vectors for objective assessment of stuttered disfluencies” by Ravi Kumar K M, Ganesan S. and “Environment Sound Classification Based on Visual Multi-Feature Fusion and GRU-AWS” By Peng N, Chen A, Zhou G, Chen W, Zhang W, Liu J, et al. Next, for the second and third features, the MFCC trajectories over time (delta MFCC, the first derivative is referred to as delta MFCC) and the second-order delta of MFCC (the second derivative is referred as delta-delta MFCC) are computed. Therefore, in one embodiment, there are 3 channels of input features that could be considered a color image (i.e., red—green-blue in the computer vision field). These three features, i.e., MFCC, derivative of MFCC, and secondary derivative of MFCC, are inputted to the first layer of the model in the form of images, so as to train the model. In an embodiment, EfficientNet is used as a main architecture for training the model using transfer learning.

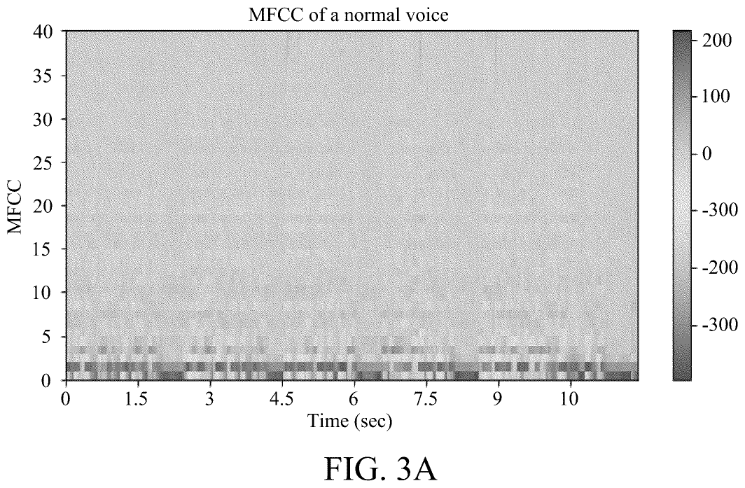

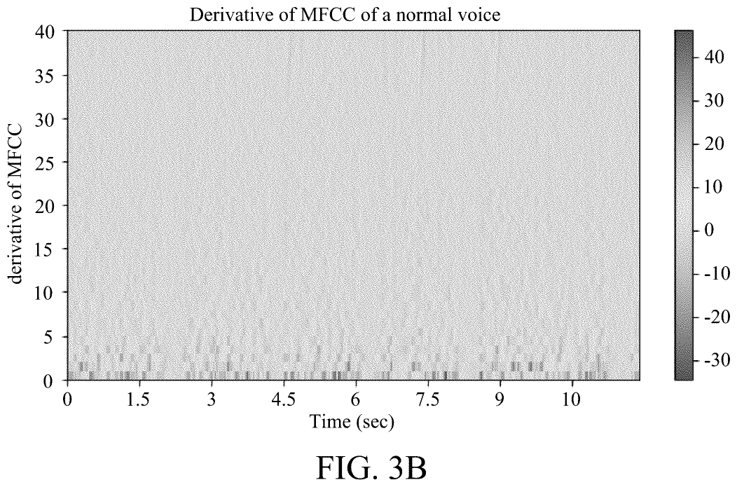

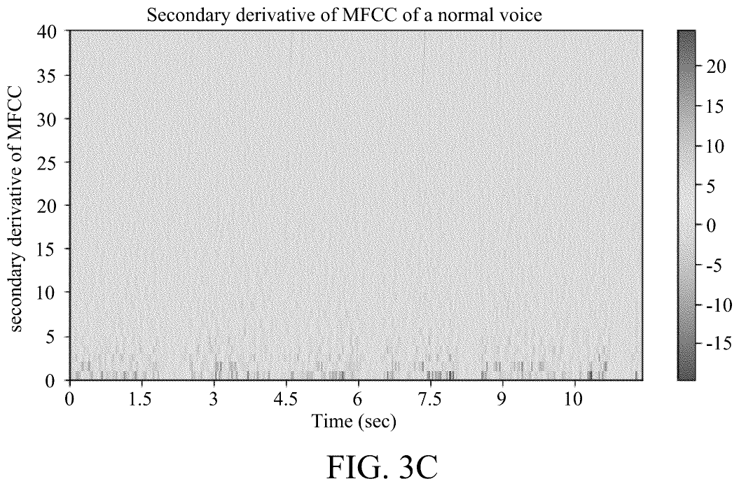

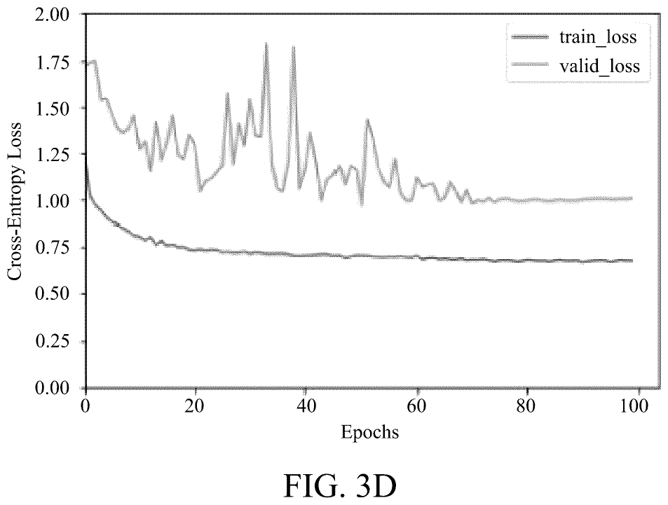

As shown in FIGS. 3A through 3C, which illustrate visual features of normal voice sample after MFCC conversion process. FIG. 3A represent a visual features of normal voice sample after MFCC conversion process; FIG. 3B shows a visual features after MFCC conversion process, representing a feature of derivative of MFCC, i.e., delta MFCC; FIG. 3C shows a visual features after MFCC conversion process, representing a feature of secondary derivative of MFCC, i.e., delta-delta MFCC.

Next, different CNN architectures, such as EfficientNet-B0 to B6, SENet154, Se_resnext101_32 x4d, and se_resnet152 models, from the ImageNet data set that have been pretrained for transfer learning are used. In an embodiment, the transfer learning may be achieved by utilizing the methodology disclosed in the document “A study on CNN transfer learning for image classification” by Hussain M, Bird J, Faria D. In an embodiment, EfficientNet-B0 to B6, SENet154, Se_resnext101_32x4d, and se_resnet152 models may be achieved by utilizing the methodology disclosed in the documents “EfficientNet: Rethinking model scaling for convolutional neural networks” by Tan M, Le Q. and “Squeeze-and-excitation networks” by Hu J, Shen L, Sun G.

Because CNNs have distinct feature representation—related characteristics, among which the lower layers provide general feature-extraction capabilities and the higher layers include information that is increasingly more specific to the original classification task. This allows verbatim reuse of the generalized feature-extraction and representation of the lower CNN layers; the higher layers are fine-tuned toward secondary problem domains with characteristics related to the original.

In one embodiment, pathological conditions are classified into 2, 3, 4, or 5 different classifications, and then input to the CNN to train the CNN, that is, two classifications (normal voice; adductor spasmodic dysphonia plus organic vocal fold lesions plus unilateral vocal paralysis plus vocal atrophy), three classifications (normal voice; adductor spasmodic dysphonia; organic vocal fold lesions plus unilateral vocal paralysis plus vocal atrophy), four classifications (normal voice; adductor spasmodic dysphonia; organic vocal fold lesions; unilateral vocal paralysis plus vocal atrophy), or five classifications (normal voice; adductor spasmodic dysphonia; organic vocal fold lesions; unilateral vocal paralysis; vocal atrophy). In an embodiment, the voice samples are first classified according to clinical diagnosis and then marked as ground truth before training the CNN. For the final prediction of an input instance, for example, the maximum probability is used to obtain the label. For instance, there are five classifications and the probability of each of a sample may be A: 0.6, B 0.1, C 0.2, D 0.05, E 0.05. Since A has the highest probability, the prediction (lable) of the sample is A.

In terms of hyperparameter settings for fine-tuning, among the training set, 474 of 593 samples (79.9%) are used for initial training and 119 of 593 samples (20.1%) (hereinafter validation set) are used for validation. In an embodiment, after initial training, the validation set is used to validate an initially trained model, and the hyper-parameters may be adjusted depending up a validation result so as to retrain the model, and then the validation set may be used to validate a retrained model.

In addition, the dropout function is added and different data augmentation methods are adopted to prevent the model from overfitting in the data set. In an embodiment, the dropout function and the data augmentation may be performed by utilizing the methodology disclosed in the documents “The effectiveness of data augmentation in image classification using deep learning” by Perez L, Wang J. and “Towards dropout training for convolutional neural networks” by Wu H, Gu X.). In an embodiment, the dropout rate is set at 0.25-0.5 for regularization.

Next, the model according to the present disclosure is trained using, e.g., minibatches of 32, which are selected based on memory consumption. In an embodiment, the minibatch may be performed by utilizing the methodology disclosed in the document “Mini-batch serialization: CNN training with inter-layer data reuse” by Lym S, Behroozi A, Wen W, Li G, Kwon Y, Erez M.

Meanwhile, the learning rate is tuned based on cosine annealing and a 1—cycle policy strategy. In an embodiment, the learning rate and the 1-cycle policy strategy may be performed by utilizing the methodology disclosed in the documents “Snapshot ensembles: train 1, get M for free” by Huang G, Li Y, Pleiss G, Liu Z, Hoperoft J, Weinberger K. and “A disciplined approach to neural network hyper-parameters: Part 1—learning rate, batch size, momentum, and weight decay” by Smith L. By using the cosine annealing schedule, the model repeatedly fits the gradient to the local minimum.

Furthermore, the model according to the present disclosure is trained end-to-end using an algorithm such as Adam optimization algorithm, and it optimizes the cross-entropy as a loss function. In an embodiment, the Adam optimization algorithm may be performed by utilizing the methodology disclosed in the document “Adam: A method for stochastic optimization” by Kingma D, Ba J.

For different classification problems in the model head, a SoftMax layer is applied as an output layer for multiclass classification or a sigmoid layer for binary classification.

Finally, the model is assembled by average output probability to receive more robust results to minimize the bias of prediction error to improve the prediction accuracy of the CNN models. In an embodiment, the assembles may be performed by using the methodology disclosed in the document “Snapshot ensembles: train 1, get M for free” by Huang G, Li Y, Pleiss G, Liu Z, Hoperoft J, Weinberger K. In an embodiment, the probability of each of the sub-models in EfficientNet is calculated respectively and then averaged to determine the final predicted value.

### Statistical Analysis

The effectiveness of the model according to the present disclosure is evaluated by several metrics, including accuracy, sensitivity, specificity, F1 score, receiver-operating characteristic (ROC) curve, and area under the curve (AUC). All metrics may be calculated using Python.

### Results

Voice samples according to the present disclosure are composed of a sustained vowel sound and a continuous essay speech. The whole voice sample (i.e., the vowel sound and essay) is applied during subsequent machine learning because the combination of the vowel sound and essay group (F1 score=0.65) achieves better F1 scores than the vowel sound group (F1 score=0.54) and the essay group (F1 score=0.57).

Referring to FIG. 3D, it shows the changes in the loss function value over the training and validation sets, which demonstrates that the model according to the present disclosure could converge after running the optimization for a number of epochs. In FIG. 3D, the upper and lower curves refer to the loss function value for the validation sets and training sets, respectively.

Referring to Table 2, it presents the training results for the different classification conditions, including two classifications (normal voice; adductor spasmodic dysphonia plus organic vocal fold lesions plus unilateral vocal paralysis plus vocal atrophy), three classifications (normal voice; adductor spasmodic dysphonia; organic vocal fold lesions plus unilateral vocal paralysis plus vocal atrophy), four classifications (normal voice; adductor spasmodic dysphonia; organic vocal fold lesions; unilateral vocal paralysis plus vocal atrophy), five classifications (normal voice; adductor spasmodic dysphonia; organic vocal fold lesions; unilateral vocal paralysis; vocal atrophy), which are used to trained the CNN model.

TABLE 2
Performance of the artificial intelligence model for classifying
voice disorders under different classification conditions.

| | | | | | Average area |
|---|---|---|---|---|---|
| | | | Accuracy, | F1 | under the curve |
| Class | Sensitivity | Specificity | % | score | (AUC) values |
| 2 | 0.99 | 0.84 | 95.3 | 0.97 | 0.98 |
| 3 | 0.82 | 0.93 | 91.2 | 0.80 | 0.96 |
| 4 | 0.75 | 0.89 | 71.0 | 0.75 | 0.88 |
| 5 | 0.66 | 0.91 | 66.9 | 0.66 | 0.85 |

In the model according to the present disclosure, the two classifications condition could equally distinguish pathological voices from normal voices. As shown in Table 2, the accuracy of pathological voice detection reaches 95.3%; the sensitivity is 99%, specificity is 84%, and AUC is 0.98. By the three classifications condition, it could identify adductor spasmodic dysphonia patients from those with other vocal fold pathologies. As shown in Table 2, the accuracy is 91.2%, sensitivity is 82%, specificity is 93%, and AUC is 0.96. By the four classifications condition, vocal atrophy and unilateral vocal paralysis could be clinically grouped as “glottis insufficiency.” As shown in Table 2, the accuracy is 71.0%, sensitivity is 75%, specificity is 89%, and AUC is 0.88. By the five classifications condition, as shown in Table 2, the accuracy is 66.9%, sensitivity is 66%, specificity is 91%, and AUC is 0.85.

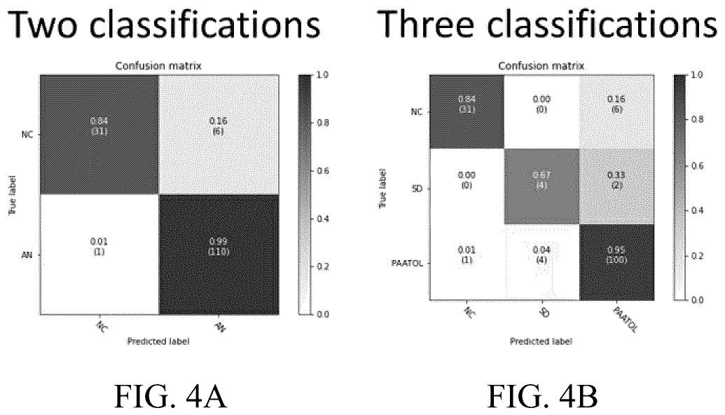

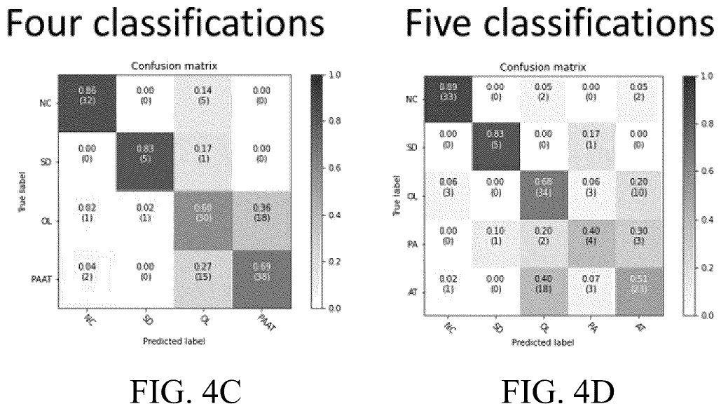

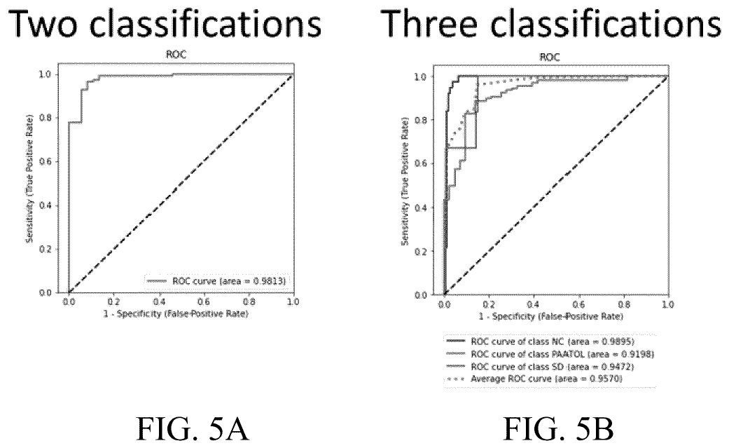

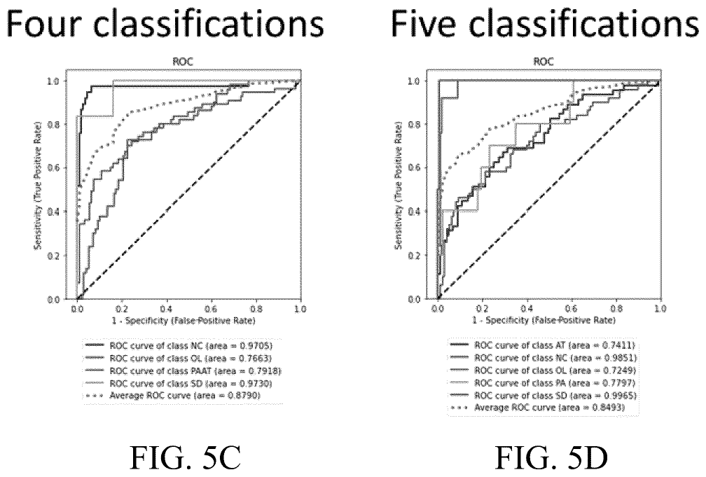

Referring to FIGS. 4A-4D, it shows the confusion matrix of these results. Referring to FIGS. 5A-5D, it shows the receiver-operating characteristic (ROC) curves of these results. In FIGS. 4A-4D and FIGS. 5A-5D, a dotted line represents average ROC curve; NC represents normal voice; AN represents pathological voice; SD represents adductor spasmodic dysphonia; PAATOL represents unilateral vocal paralysis plus vocal atrophy plus organic vocal fold lesions; OL represents organic vocal fold lesions; PAAT represents unilateral vocal paralysis plus vocal atrophy; PA represents unilateral vocal paralysis; and AT represents vocal atrophy. Table 3 shows the detail.

TABLE 3

| | Class | Class | Class | Class |
|---|---|---|---|---|
| | 2 | 3 | 4 | 5 |
| normal voice (NC) | NC | NC | NC | NC |
| adductor spasmodic dysphonia (SD) | AN | SD | SD | SD |
| organic vocal fold lesions (OL) | | PAATOL | OL | OL |
| unilateral vocal paralysis (PA) | | | PAAT | PA |
| vocal atrophy (AT) | | | | AT |

FIGS. 4A and 5A show the confusion matrix and the receiver-operating characteristic (ROC) curves of the two classifications, respectively. FIGS. 4B and 5B show the confusion matrix and the receiver-operating characteristic (ROC) curves of the three classifications, respectively. FIGS. 4C and 5C show the confusion matrix and the receiver-operating characteristic (ROC) curves of the four classifications, respectively. FIGS. 4D and 5D show the confusion matrix and the receiver-operating characteristic (ROC) curves of the five classifications, respectively.

Based on Tables 2 and 3, FIGS. 4A-4D and FIGS. 5A-5D, the model according to the present disclosure distinguishes, with high specificity (91%), different pathological voices (adductor spasmodic dysphonia; organic vocal fold lesions; unilateral vocal paralysis; vocal atrophy) attributable to common vocal diseases based on voice alone, i.e., the vowel sound and essay. Moreover, the model according to the present disclosure distinguishes normal voice (NC) and adductor spasmodic dysphonia (SD) with AUC values: 0.985 and 0.997, respectively, for the five-classification condition.

Referring to Table 4, it presents four ear, nose, and throat (ENT) specialists to identify vocal fold pathology by voice using these 5 classifications. In the table, the accuracy rates are 60.1% and 56.1% for the 2 laryngologists and 51.4% and 43.2% for the 2 general ENT specialists.

TABLE 4
Comparison of the performance for a 5-classification condition
by our artificial intelligence model and 4 human experts.

| Test participants | Sensitivity | Specificity | Accuracy, % |
|---|---|---|---|
| Deep learning model | 0.66 | 0.91 | 66.9 |
| Laryngologist A (11 years | 0.61 | 0.89 | 60.1 |
| of experience) | | | |
| Laryngologist B (10 years | 0.63 | 0.88 | 56.1 |
| of experience) | | | |
| General ENT C (8 years | 0.54 | 0.88 | 51.4 |
| of experience) | | | |
| General ENT D (14 years | 0.42 | 0.85 | 43.2 |
| of experience) | | | |

Based on Tables 2 and 4, the overall accuracy of the model according to the present disclosure is better than that of all ENT specialists participating.

Comparing the accuracy of each classification, it is noted that artificial intelligence is markedly better than laryngologists when identifying organic vocal fold lesions (artificial intelligence, 68%; laryngologist A, 60%; laryngologist B, 24%). The reason that the organic vocal fold lesions is difficult for humans to identify is the difference in the vibration pattern of Organic vocal fold lesions, unilateral vocal paralysis, and vocal atrophy could only be observed by high-speed video and multislice digital videokymography. For example, in the case of organic vocal fold lesions during vibration, the lesion divided the fold into 2 oscillators; in the case of unilateral vocal paralysis, vibrating frequencies were different between the normal vocal fold and paralysis vocal fold; and vocal atrophy will show a breakdown of vibration with a visible repetition in the loss of normal vibration every few glottal cycles. Therefore, the method and system for pathological voice recognition and a non-transitory computer-readable storage medium storing computer-readable instructions according to this application can recognize various vocal fold pathologies, such as unilateral vocal paralysis, adductor spasmodic dysphonia, vocal atrophy, and organic vocal fold lesions, based on voice signal of a person, because different vibration patterns of the vocal fold would be caused by vocal fold pathologies.

In addition, four human specialists require 40-80 minutes to identify 148 voice samples of the test set; however, the model accordingly to the present disclosure requires 30 seconds to perform the same task.

In conclusion, the present disclosure shows that voice alone could be used for common vocal fold disease recognition using a deep learning application after training with pathological voice database of the present disclosure. In one embodiment, adductor spasmodic dysphonia, organic vocal fold lesions, unilateral vocal paralysis, and vocal atrophy could be recognized, which could increase the potential of this approach to be more beneficial than simply distinguishing a pathological voice from a normal voice. This approach shows clinical potential for use during general screening of different vocal fold diseases based on voice and could be included in quick evaluations during general health examinations. It could also be used for telemedicine in remote regions that lack laryngoscopy services in primary care units. Overall, it could support physicians during prescreening of cases by allowing for invasive examinations to be performed only for cases involving problems with automatic recognition or listening and for professional analyses of other clinical examination results that reveal doubts about the presence of pathologies.

The techniques described above may be implemented, for example, in hardware, one or more computer programs tangibly stored on one or more computer-readable media, firmware, or any combination thereof. The techniques described above may be implemented in one or more computer programs executing on (or executable by) a programmable computer including any combination of any number of the following: a processor, a storage medium readable and/or writable by the processor (including, for example, volatile and non-volatile memory and/or storage elements), an input device, and an output device. Program code may be applied to input entered using the input device to perform the functions described and to generate output using the output device.

Each computer program within the scope of the claims below may be implemented in any programming language, such as assembly language, machine language, a high-level procedural programming language, or an object-oriented programming language. The programming language may, for example, be a compiled or interpreted programming language. Each such computer program may be implemented in a computer program product tangibly embodied in a machine-readable storage device for execution by a computer processor. Method steps of the invention may be performed by one or more computer processors executing a program tangibly embodied on a computer-readable medium to perform functions of the invention by operating on input and generating output. Suitable processors include, by way of example, both general and special purpose microprocessors. Generally, the processor receives (reads) instructions and data from a memory (such as a read-only memory and/or a random-access memory) and writes (stores) instructions and data to the memory. Storage devices suitable for tangibly embodying computer program instructions and data include, for example, all forms of non-volatile memory, such as semiconductor memory devices, including EPROM, EEPROM, and flash memory devices; magnetic disks such as internal hard disks and removable disks; magneto-optical disks; and CD-ROMs. Any of the foregoing may be supplemented by, or incorporated in, specially-designed ASICs (application-specific integrated circuits) or FPGAs (Field-Programmable Gate Arrays). A computer can generally also receive (read) programs and data from, and write (store) programs and data to, a non-transitory computer-readable storage medium such as an internal disk (not shown) or a removable disk.

The present disclosure has been described with exemplary embodiments to illustrate the features and efficacies of the present disclosure, but not intend to limit the scope of the present disclosure. The present disclosure without departing from the scope of the premise can make various changes and modifications by a person skilled in the art. However, any equivalent change and modification accomplished according to the present disclosure should be considered as being covered in the scope of the present disclosure. The scope of the disclosure should be defined by the appended claims.

What is claimed is:

1. A method for pathological voice recognition, the method comprising:
capturing a voice signal;
processing the voice signal using Mel Frequency Cepstral Coefficients (MFCC) algorithm to obtain an MFCC spectrogram;
extracting features from the MFCC spectrogram; and
predicting a pathological condition of the voice signal based on the features of the MFCC spectrogram of the voice signal by a deep learning model.

2. The method of claim 1, further comprising:
capturing a plurality of voice samples into a database;
dividing the plurality of voice samples into a training set and a testing set;
processing the training set of the plurality of voice samples using the MFCC algorithm to obtain a plurality of MFCC spectrograms;
extracting a plurality of features from the plurality of MFCC spectrograms of the training set of the voice samples; and
inputting the plurality of features into the deep learning model to train the deep learning model, wherein the plurality of features comprises MFCC spectrogram, delta MFCC spectrogram, and/or second-order delta MFCC spectrogram.

3. The method of claim 2, wherein each of the plurality of voice samples includes a sustained vowel sound followed by a continuous speech.

4. The method of claim 2, further comprising:
training the deep learning model by classifying the training set of the voice samples into two classifications,
wherein the two classifications include normal voices and a group of adductor spasmodic dysphonia, organic vocal fold lesions, unilateral vocal paralysis, and vocal atrophy.

5. The method of claim 2, further comprising:
training the deep learning model by classifying the training set of the voice samples into three classifications,
wherein the three classifications include normal voices, adductor spasmodic dysphonia, and a group consist of organic vocal fold lesions, unilateral vocal paralysis, and vocal atrophy.

6. The method of claim 2, further comprising:
training the deep learning model by classifying the training set of the voice samples into four classifications,
wherein the four classifications include normal voices, adductor spasmodic dysphonia, organic vocal fold lesions, and a group consist of unilateral vocal paralysis and vocal atrophy.

7. The method of claim 2, further comprising:
training the deep learning model by classifying the training set of the voice samples into five classifications,
wherein the five classifications include normal voices, adductor spasmodic dysphonia, organic vocal fold lesions, unilateral vocal paralysis, and vocal atrophy.

8. The method of claim 2, further comprising:
training the deep learning model by adding a dropout function, using minibatches, tuning a learning rate based on cosine annealing and a 1-cycle policy strategy, and applying a SoftMax layer as an output layer; and
assembling the trained deep learning model by average output probability.

9. The method of claim 2, wherein the extracting the plurality of features from the plurality of MFCC spectrograms of the training set of the voice samples comprises: using pre-emphasis, windowing, fast Fourier transform, Mel filtering, nonlinear transformation, and/or discrete cosine transform to extract the plurality of features therefrom.

10. The method of claim 9, wherein the plurality of features comprises MFCC, delta MFCC, and/or second-order delta MFCC.

11. A non-transitory computer-readable storage medium storing computer-readable instructions that, when executed, cause a system to perform the method of claim 1.

12. A system for pathological voice recognition, the system comprising:
a transducer configured to capture a voice signal;
a processor including a deep learning model, configured to;
process the voice signal using Mel Frequency Cepstral Coefficients (MFCC) algorithm to obtain an MFCC spectrogram;
extract features from the MFCC spectrogram; and
predict a pathological condition of the voice signal based on the features of the MFCC spectrogram of the voice signal by the deep learning model.

13. The system of claim 12, further comprising:
a database configured to receive a plurality of voice samples captured by the transducer;
wherein the processor is configured to:
divide the plurality of voice samples into a training set and a testing set;
process the training set of the voice samples using MFCC algorithm to obtain a plurality of MFCC spectrograms;
extract a plurality of features from the plurality of MFCC spectrograms of the training set of the voice samples; and
input the plurality of features into the deep learning model to train the deep learning model, wherein the plurality of features comprises MFCC spectrogram, delta MFCC spectrogram, and/or second-order delta MFCC spectrogram.

14. The system of claim 13, wherein each of the plurality of voice samples includes a sustained vowel sound followed by a continuous speech.

15. The system of claim 13, wherein the processor is further configured to:
train the deep learning model by classifying the training set of the voice samples into two classifications,
wherein the two classifications include normal voices and a group of adductor spasmodic dysphonia, organic vocal fold lesions, unilateral vocal paralysis, and vocal atrophy.

16. The system of claim 13, wherein the processor is further configured to:
train the deep learning model by classifying the training set of the voice samples into three classifications,
wherein the three classifications include normal voices, adductor spasmodic dysphonia, and a group consist of organic vocal fold lesions, unilateral vocal paralysis, and vocal atrophy.

17. The system of claim 13, wherein the processor is further configured to:
train the deep learning model by classifying the training set of the voice samples into four classifications,
wherein the four classifications include normal voices, adductor spasmodic dysphonia, organic vocal fold lesions, and a group consist of unilateral vocal paralysis and vocal atrophy.

18. The system of claim 13, wherein the processor is further configured to:
train the deep learning model by classifying the training set of the voice samples into five classifications,
wherein the five classifications include normal voices, adductor spasmodic dysphonia, organic vocal fold lesions, unilateral vocal paralysis, and vocal atrophy.

19. The system of claim 13, wherein the processor is further configured to:
train the deep learning model by adding a dropout function, using minibatches, tuning a learning rate based on cosine annealing and a 1-cycle policy strategy, and applying a SoftMax layer as an output layer; and
assemble the trained deep learning model by average output probability.

20. The system of claim 13, wherein the processor is further configured to use pre-emphasis, windowing, fast Fourier transform, Mel filtering, nonlinear transformation, and/or discrete cosine transform to extract the plurality of features, wherein the plurality of features comprises MFCC, delta MFCC, and/or second-order delta MFCC.
